(19) United States
(12) Patent Application Publication
(10) Pub. No.: US 2006/0058651 A1
(43) Pub. Date: Mar. 16, 2006

(54) Method and apparatus for extending an ultrasound image field of view

(76) Inventors: Richard Yung Chiao, Issaquah, WA (US); Steven Charles Miller, Waukesha, WI (US)

Correspondence Address: Dean D. Small;Armstrong Teasdale LLP, Suite 2600, One Metropolitan Square, St. Louis, MO, 63102, US

(21) Appl. No.: 10/917,749

(22) Filed: Aug. 13, 2004

(51) Int. Cl. A61B 8/00

(52) U.S. Cl. 600/437

(57) ABSTRACT

A method and apparatus for extending a field of view of a medical imaging system is provided. The method includes scanning a surface of an object using an ultrasound transducer, obtaining a plurality of 3-D volumetric data sets, at least one of the plurality of data sets having a portion that overlaps with another of the plurality of data sets, and generating a panoramic 3-D volume image using the overlapping portion to register spatially adjacent 3-D volumetric data sets.

### BACKGROUND OF THE INVENTION

This invention relates generally to ultrasound systems and, more particularly, to methods and apparatus for acquiring and combining images in ultrasound systems.

Traditional 2-D ultrasound scans capture and display a single image slice of an object at a time. The position and orientation of the ultrasound probe at the time of the scan determines the slice imaged. At least some known ultrasound systems, for example, an ultrasound machine or scanner, are capable of acquiring and combining 2-D images into a single panoramic image. Current ultrasound systems also have the capability to acquire image data to create 3-D volume images. 3-D imaging may allow for facilitation of visualization of 3-D structures that is clearer in 3-D than as a 2-D slice, visualization of reoriented slices within the body that may not be accessible by direct scanning, guidance and/or planning of invasive procedures, for example, biopsies and surgeries, and communication of improved scan information with colleagues or patients.

A 3-D ultrasound image may be acquired as a stack of 2-D images in a given volume. An exemplary method of acquiring this stack of 2-D images is to manually sweep a probe across a body such that a 2-D image is acquired at each position of the probe. The manual sweep may take several seconds, so this method produces “static” 3-D images. Thus, although 3-D scans image a volume within the body, the volume is a finite volume, and the image is a static 3-D representation of the volume.

### BRIEF DESCRIPTION OF THE INVENTION

In one embodiment, a method and apparatus for extending a field of view of a medical imaging system is provided. The method includes scanning a surface of an object using an ultrasound transducer, obtaining a plurality of 3-D volumetric data sets, at least one of the plurality of data sets having a portion that overlaps with another of the plurality of data sets, and generating a panoramic 3-D volume image using the overlapping portion to register spatially adjacent 3-D volumetric data sets.

In another embodiment, an ultrasound system is provided. The ultrasound system includes a volume rendering processor configured to receive image data acquired as at least one of a plurality of scan planes, a plurality of scan lines, and volumetric data sets, and a matching processor configured to combine projected volumes into a combined volume image in real-time.

### BRIEF DESCRIPTION OF THE DRAWINGS

FIG. 1 is a block diagram of an ultrasound system in accordance with one exemplary embodiment of the present invention;

FIG. 2 is a block diagram of an ultrasound system in accordance with another exemplary embodiment of the present invention;

FIG. 3 is a perspective view of an image of an object acquired by the systems of FIGS. 1 and 2 in accordance with an exemplary embodiment of the present invention; and

FIG. 4 is a perspective view of an exemplary scan using an array transducer to produce a panoramic 3-D image according to various embodiments of the present invention.

### DETAILED DESCRIPTION OF THE INVENTION

As used herein, the term “real time” is defined to include time intervals that may be perceived by a user as having little or substantially no delay associated therewith. For example, when a volume rendering using an acquired ultrasound dataset is described as being performed in real time, a time interval between acquiring the ultrasound dataset and displaying the volume rendering based thereon may be in a range of less than about one second. This reduces a time lag between an adjustment and a display that shows the adjustment. For example, some systems may typically operate with time intervals of about 0.10 seconds. Time intervals of more than one second also may be used.

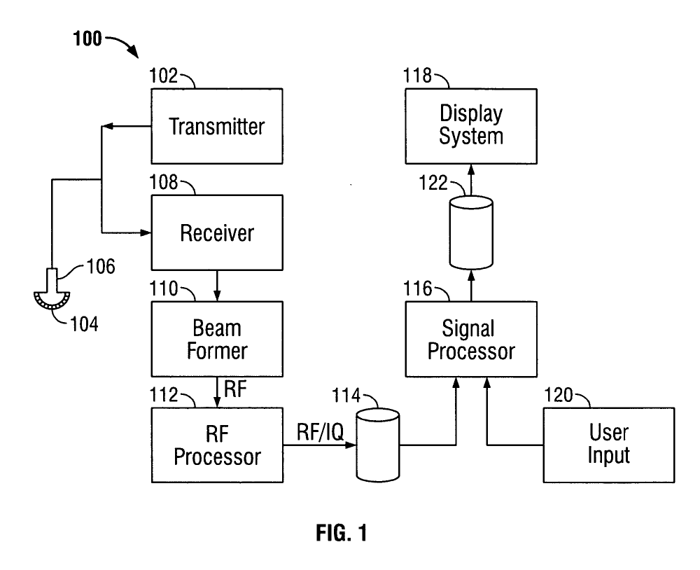

FIG. 1 is a block diagram of an ultrasound system in accordance with one exemplary embodiment of the present invention. The ultrasound system 100 includes a transmitter 102 that drives an array of elements 104 (e.g., piezoelectric crystals) within or formed as part of a transducer 106 to emit pulsed ultrasonic signals into a body or volume. A variety of geometries may be used and one or more transducers 106 may be provided as part of a probe (not shown). The pulsed ultrasonic signals are back-scattered from density interfaces and/or structures, for example, blood cells or muscular tissue, to produce echoes that return to elements 104. The echoes are received by a receiver 108 and provided to a beamformer 110. The beamformer performs beamforming on the received echoes and outputs a RF signal. A RF processor 112 then processes the RF signal. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and prepare frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation.

The ultrasound system 100 may continuously acquire ultrasound information at a frame rate that exceeds twenty frames per second, which is the approximate perception rate of the human eye. The acquired ultrasound information may be displayed on display system 118 at a slower frame-rate. An image buffer 122 may be included for storing processed frames of acquired ultrasound information that are not scheduled to be displayed immediately. In an exemplary embodiment, image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of ultrasound system 100. The user input device 120 may be any suitable device and/or user interface for receiving user inputs to control, for example, the type of scan or type of transducer to be used in a scan.

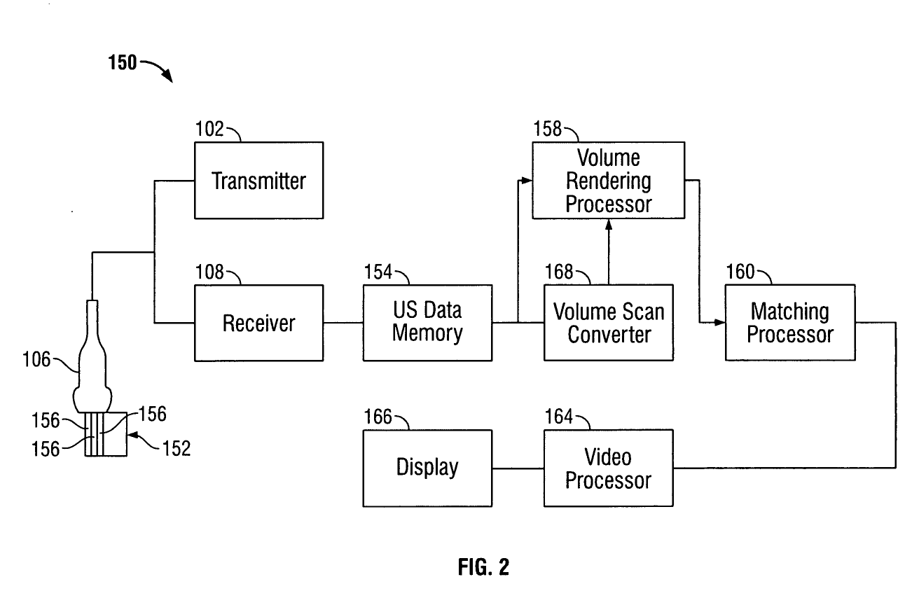

FIG. 2 is a block diagram of an ultrasound system 150 in accordance with another exemplary embodiment of the present invention. The system includes transducer 106 connected to transmitter 102 and receiver 108. Transducer 106 transmits ultrasonic pulses and receives echoes from structures inside of a scanned ultrasound volume 410 (shown in FIG. 4). A memory 154 stores ultrasound data from receiver 108 derived from scanned ultrasound volume 410. Volume 410 may be obtained by various techniques (e.g., 3-D scanning, real-time 3-D imaging, volume scanning, 2-D scanning with an array of elements having positioning sensors, freehand scanning using a Voxel correlation technique, and/or 2-D or matrix array transducers).

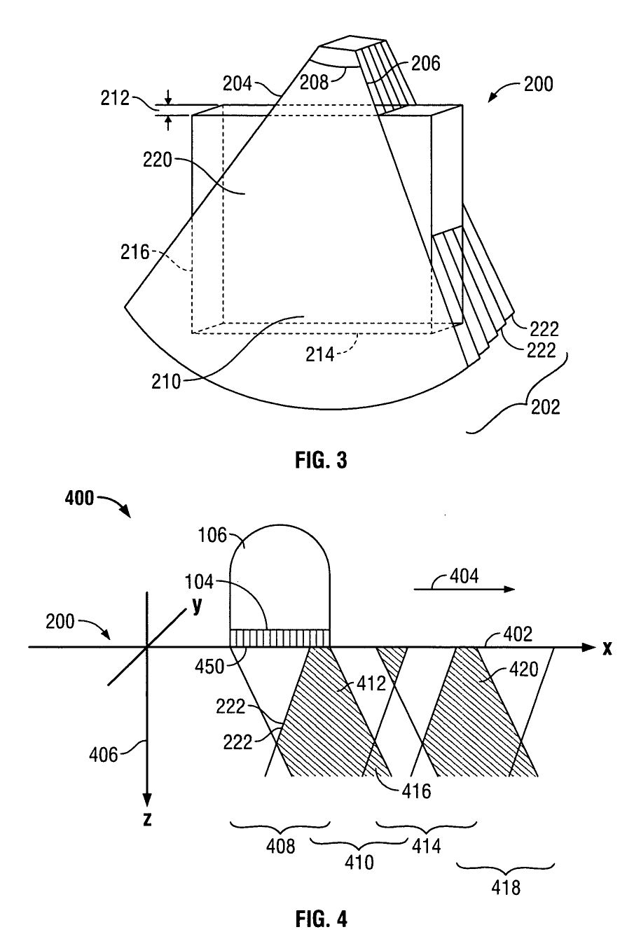

Transducer 106 may be moved linearly or arcuately to obtain a panoramic 3-D image while scanning a volume. At each linear or arcuate position, transducer 106 obtains a plurality of scan planes 156 as transducer 106 is moved. Scan planes 156 are stored in memory 154, then transmitted to a volume rendering processor 158. Volume rendering processor 158 may receive 3-D image data sets directly. Alternatively, scan planes 156 may be transmitted from memory 154 to a volume scan converter 168 for processing, for example, to perform a geometric translation, and then to volume rendering processor 158. After 3-D image data sets and/or scan planes 156 have been processed by volume rendering processor 158 the data sets and/or scan planes 156 may be transmitted to a matching processor 160 and combined to produce a combined panoramic volume with the combined panoramic volume transmitted to a video processor 164. It should be understood that volume scan converter 168 may be incorporated within volume rendering processor 158. In some embodiments, transducer 106 may obtain scan lines instead of scan planes 156, and memory 154 may store scan lines obtained by transducer 106 rather than scan planes 156. Volume scan converter 168 may process scan lines obtained by transducer 106 rather than scan planes 156, and may create data slices that may be transmitted to volume rendering processor 158. The output of volume rendering processor 158 is transmitted to matching processor 160, video processor 164 and display 166. Volume rendering processor 158 may receive scan planes, scan lines, and/or volume image data directly, or may receive scan planes, scan lines, and/or volume data through volume scan converter 168. Matching processor 160 processes the scan planes, scan lines, and/or volume data to locate common data features and combine 3-D volumes based on the common data features into real-time panoramic image data sets that may be displayed and/or further processed to facilitate identifying structures within an object 200 (shown in FIG. 3), and as described in more detail herein.

The position of each echo signal sample (Voxel) is defined in terms of geometrical accuracy (i.e., the distance from one Voxel to the next) and ultrasonic response (and derived values from the ultrasonic response). Suitable ultrasonic responses include gray scale values, color flow values, and angio or power Doppler information.

System 150 may acquire two or more static volumes at different, overlapping locations, which are then combined into a combined volume. For example, a first static volume is acquired at a first location, then transducer 106 is moved to a second location and a second static volume is acquired. Alternatively, the scan may be performed automatically by mechanical or electronic means that can acquire greater than twenty volumes per second. This method generates “real-time” 3-D images. Real-time 3-D images are generally more versatile than static 3-D because moving structures can be imaged and the spatial dimensions may be correctly registered.

FIG. 3 is a perspective view of an image of an object acquired by the systems of FIGS. 1 and 2 in accordance with an exemplary embodiment of the present invention. Object 200 includes a volume 202 defined by a plurality of sector shaped cross-sections with radial borders 204 and 206 diverging from one another at an angle 208. Transducer 106 (shown in FIGS. 1 and 2) electronically focuses and directs ultrasound firings longitudinally to scan along adjacent scan lines in each scan plane 156 (shown in FIG. 2) and electronically or mechanically focuses and directs ultrasound firings laterally to scan adjacent scan planes 156. Scan planes 156 obtained by transducer 106, and as illustrated in FIG. 1, are stored in memory 154 and are scan converted from spherical to Cartesian coordinates by volume scan converter 168. A volume comprising multiple scan planes 156 is output from volume scan converter 168 and stored in a slice memory (not shown) as a rendering region 210. Rendering region 210 in the slice memory is formed from multiple adjacent scan planes 156.

Transducer 106 may be translated at a constant speed while images are acquired, so that individual scan planes 156 are not stretched or compressed laterally relative to earlier acquired scan planes 156. It is also desirable for transducer 106 to be moved in a single plane, so that there is high correlation from each scan planes 156 to the next. However, manual scanning over an irregular body surface may result in departures from either or both of these desirable conditions. Automatic scanning and/or motion detection and 2-D image connection may reduce undesirable conditions/effects of manual scanning.

Rendering region 210 may be defined in size by an operator using a user interface or input to have a slice thickness 212, a width 214 and a height 216. Volume scan converter 168 (shown in FIG. 2) may be controlled by slice thickness setting control (not shown) to adjust the thickness parameter of a slice 222 to form a rendering region 210 of the desired thickness. Rendering region 210 defines the portion of scanned ultrasound volume 410 (shown in FIG. 4) that is volume rendered. Volume rendering processor 158 accesses the slice memory and renders along slice thickness 212 of rendering region 210. Volume rendering processor 158 may be configured to render a three dimensional presentation of the image data in accordance with rendering parameters selectable a user through user input 120.

During operation, a slice having a pre-defined, substantially constant thickness (also referred to as rendering region 210) is determined by the slice thickness setting control and is processed in volume scan converter 168. The echo data representing rendering region 210 (shown in FIG. 3) may be stored in slice memory. Predefined thicknesses between about 2 mm and about 20 mm are typical, however, thicknesses less than about 2 mm or greater than about 20 mm may also be suitable depending on the application and the size of the area to be scanned. The slice thickness setting control may include a control member, such as a rotatable knob with discrete or continuous thickness settings.

Volume rendering processor 158 projects rendering region 210 onto an image portion 220 of slice 222 (shown in FIG. 3). Following processing in volume rendering processor 158, pixel data in image portion 220 may be processed by matching processor 160, video processor 164 and then displayed on display 166. Rendering region 210 may be located at any position and oriented at any direction within volume 202. In some situations, depending on the size of the region being scanned, it may be advantageous for rendering region 210 to be only a small portion of volume 202. It will be understood that the volume rendering disclosed herein can be gradient-based volume rendering that uses, for example, ambient, diffuse, and specular components of the 3-D ultrasound data sets to render the volumes. Other components may also be used. It will also be understood that the volume renderings may include surfaces that are part of the exterior of an organ or are part of internal structures of the organ. For example, with regard to the heart, the volumes that are rendered can include exterior surfaces of the heart or interior surfaces of the heart where, for example, a catheter is guided through an artery to a chamber of the heart.

FIG. 4 is a perspective view of an exemplary scan 400 using array transducer 106 to produce a panoramic 3-D image according to various embodiments of the present invention. Array transducer 106 includes elements 104 and is shown in contact with a surface 402 of object 200. To scan object 200, array transducer 106 is swept across surface 402 in a direction 404. As array transducer 106 is moved in direction 404, (e.g., x-direction) successive slices 222 are acquired, each being slightly displaced (as a function of the speed of array transducer 106 motion and the image acquisition rate) in direction 404 from previous slice 222. The displacement between successive slice 222 is computed and slices 222 are registered and combined on the basis of the displacements to produce a 3-D volume image.

Transducer 106 may acquire consecutive volumes comprising 3-D volumetric data in a depth direction 406 (e.g., z-direction). Transducer 106 may be a mechanical transducer having a wobbling element 104 or array of elements 104 that are electrically controlled. Although the scan sequence of FIG. 4 is representative of scan data acquired using a linear transducer 106, other transducer types may be used. For example, transducer 106 may be a 2-D array transducer, which is moved by the user to acquire the consecutive volumes as discussed above. Transducer 106 may also be swept or translated across surface 402 mechanically. As transducer 106 is translated, ultrasound images of the collected data are displayed to the user such that the progress and quality of the scan may be monitored. If the user determines a portion of the scan is of insufficient quality, the user may stop the scan, selectably remove or erase data corresponding to the portion of the scan to be replaced. When restarting the scan, system 100 may automatically detect and reregister the newly acquired scan data with the volumes still in memory. If system 100 is unable to reregister the incoming image data with the data stored in memory, for example if the scan did not restart such that there is overlap between the data in memory and the newly acquired data, system 100 may identify the misregistered portion on display 166 and/or initiate a audible and/or visual alarm.

Transducer 106 acquires a first volume 408. Transducer 106 may be moved by the user at a constant or variable speed in direction 404 along surface 402 as the volumes of data are acquired. The position at which the next volume is acquired is based upon the frame rate of the acquisition and the physical movement of transducer 106. Transducer 106 then acquires a second volume 410. Volumes 408 and 410 include a common region 412. Common region 412 includes image data representative of the same area within object 200, however, the data of volume 410 has been acquired having different coordinates with respect to the data of volume 408, as common region 412 was scanned from different angles and a different location with respect to the x, y, and z directions. A third volume 414 may be acquired and includes a common region 416, which is shared with volume 410. A fourth volume 418 may be acquired and includes common region 420, which is shared with volume 414. This volume acquisition process may be continued as desired or needed (e.g., based upon the field of view of interest).

Each volume 408-418 has outer limits, which correspond to the scan boundaries of transducer 106. The outer limits may be described as maximum elevation, maximum azimuth, and maximum depth. The outer limits may be modified within predefined limits by changing, for example, scan parameters such as transmission frequency, frame rate, and focal zones.

In an alternative embodiment, a series of volume data sets of object 200 may be obtained at a series of respective times. For example, system 150 may acquire one volume data sets every 0.05 seconds. The volume data sets may be stored for later examination and/or viewed as they are obtained in real-time.

Ultrasound system 150 may display views of the acquired image data included in the 3-D ultrasound dataset. The views can be, for example, of slices of tissue in object 200. For example, system 150 can provide a view of a slice that passes through a portion of object 200. System 150 can provide the view by selecting image data from the 3-D ultrasound dataset that lies within selectable area of object 200.

It should be noted that the slice may be, for example, an inclined slice, a constant depth slice, a B-mode slice, or other cross-section of object 200 at any orientation. For example, the slice may be inclined or tilted at a selectable angle within object 200.

Exemplary embodiments of apparatus and methods that facilitate displaying imaging data in ultrasound imaging systems are described above in detail. A technical effect of detecting motion during a scan and connecting 2-D image slices and 3-D image volumes is to allow visualization of volumes larger than those volume images that can be generated directly. Joining 3-D image volumes into panoramic 3-D image volumes in real-time facilitates managing image data for visualizing regions of interest in a scanned object.

It will be recognized that although the system in the disclosed embodiments comprises programmed hardware, for example, software executed by a computer or processor-based control system, it may take other forms, including hardwired hardware configurations, hardware manufactured in integrated circuit form, firmware, among others. It should be understood that the matching processor disclosed may be embodied in a hardware device or may be embodied in a software program executing on a dedicated or shared processor within the ultrasound system or may be coupled to the ultrasound system.

The above-described methods and apparatus provide a cost-effective and reliable means for facilitating viewing ultrasound data in 2-D and 3-D using panoramic techniques in real-time. More specifically, the methods and apparatus facilitate improving visualization of multi-dimensional data. As a result, the methods and apparatus described herein facilitate operating multi-dimensional ultrasound systems in a cost-effective and reliable manner.

Exemplary embodiments of ultrasound imaging systems are described above in detail. However, the systems are not limited to the specific embodiments described herein, but rather, components of each system may be utilized independently and separately from other components described herein. Each system component can also be used in combination with other system components.

While the invention has been described in terms of various specific embodiments, those skilled in the art will recognize that the invention can be practiced with modification within the spirit and scope of the claims.

What is claimed is:

1. A method for extending a field of view of a medical imaging system, said method comprising:
scanning a surface of an object using an ultrasound transducer;
obtaining a plurality of 3-D volumetric data sets, at least one of the plurality of data sets having a portion that overlaps with another of the plurality of data sets; and
generating a panoramic 3-D volume image using the overlapping portion to register spatially adjacent 3-D volumetric data sets.

2. A method in accordance with claim 1 wherein scanning a surface of an object comprises scanning a surface of the object to obtain a plurality of 2-D scan planes of the object.

3. A method in accordance with claim 2 further comprising combining the plurality of 3-D volumetric data sets using at least one of the plurality of 2-D scan planes from each 3-D volumetric data set to be combined to register the combined 3-D volumetric data sets.

4. A method in accordance with claim 1 wherein scanning a surface of an object comprises scanning a surface of the object using a 2-D array transducer.

5. A method in accordance with claim 1 wherein scanning a surface of an object comprises sweeping an ultrasound transducer across the surface of the object.

6. A method in accordance with claim 1 wherein scanning a surface of an object comprises sweeping an ultrasound transducer across the surface of the object manually.

7. A method in accordance with claim 1 wherein scanning a surface of an object comprises detecting movement of the ultrasound transducer during a scan relative to an initial transducer position.

8. A method in accordance with claim 1 wherein scanning a surface of an object comprises:
visually monitoring the quality of the scan on a display;
stopping the scan if the quality of at least a portion of the scan is less than a threshold quality, as determined by the user;
rescanning the portion of the scan; and
reregistering the overlapping 3-D data sets.

9. A method in accordance with claim 7 wherein detecting movement of the ultrasound transducer comprises detecting movement of the ultrasound transducer at least one of electro-magnetically, electro-mechanically and inertially.

10. A method in accordance with claim 7 further comprising combining adjacent ones of the plurality of 3-D volumetric data sets using the detected movement of the ultrasound transducer.

11. A method in accordance with claim 1 further comprising combining adjacent ones of the plurality of 3-D volumetric data sets using at least two identified features of overlapping portions of each 3-D volumetric data set.

12. A method in accordance with claim 1 further comprising combining adjacent ones of the plurality of 3-D volumetric data sets using at least one 2-D slice generated from a common volume of adjacent ones of the plurality of 3-D volumetric data sets.

13. A method in accordance with claim 12 further comprising generating at least one of an inclined slice, a constant depth slice, and a B-mode slice from a common volume of adjacent ones of the plurality of 3-D volumetric data sets.

14. An ultrasound system comprising:
a volume rendering processor configured to receive image data acquired as at least one of a plurality of scan planes, a plurality of scan lines, and volumetric data sets; and
a matching processor configured to combine projected volumes into a combined volume image in real-time.

15. An ultrasound system in accordance with claim 14 further comprising a volume scan converter configured to convert scan planes from a spherical coordinate system to a Cartesian coordinate system.

16. An ultrasound system in accordance with claim 14 further comprising a volume scan converter configured to receive at least one of scan planes, scan lines, and/or volume image data.

17. An ultrasound system in accordance with claim 14 wherein said volume rendering processor is configured to render a three dimensional representation of the image data.

18. An ultrasound system in accordance with claim 14 wherein said volume rendering processor is configured to render a slice of a 3-D image dataset to facilitate matching features of the 3-D image dataset with a rendered slice from another 3-D image dataset.

19. An ultrasound system in accordance with claim 15 wherein said rendered slice comprises at least one of an inclined slice, a constant depth slice, a B-mode slice, and a cross-section having a selectable orientation.

20. An ultrasound system comprising:
a volume rendering processor configured to receive image data provided as at least one of a plurality of scan planes, a plurality of scan lines, and volumetric data sets, said volume rendering processor further configured to render a slice of a 3-D image dataset to allow matching features of the 3-D image dataset with a rendered slice from another 3-D image dataset; and
a matching processor configured to combine projected volumes into a combined volume image in real-time.

21. An ultrasound system in accordance with claim 20 further comprising a volume scan converter configured to convert ultrasound image data from a spherical coordinate system to a Cartesian coordinate system;

22. An ultrasound system in accordance with claim 20 wherein said rendered slice comprises at least one of an inclined slice, a constant depth slice, a B-mode slice, and a cross-section at a selectable orientation.

23. An ultrasound system in accordance with claim 20 wherein said combined volume image is a panoramic 3-D image.
